(19) United States
(12) Patent Application Publication
(10) Pub. No.: US 2007/0231361 A1
(43) Pub. Date: Oct. 4, 2007

(54) Use of Fatty Acids to Inhibit the Growth of Aneurysms

(75) Inventors: David Brin, West Newbury, MA (US); Jack Chu, Santa Rosa, CA (US); Prema Ganesan, San Francisco, CA (US); Dianne Judd, Minneapolis, MN (US); Michel Letort, Prevessins (FR); Patrice Tremble, Santa Rosa, CA (US); Eugene Tedeschi, Santa Rosa, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/277,637

(22) Filed: Mar. 28, 2006

(51) Int. Cl. A61K 31/202

(52) U.S. Cl. 424/423; 514/560

(57) ABSTRACT

Methods of treating an aneurysm in a patient in need thereof are provided. The methods comprise delivering to a treatment site an effective amount of a fatty acid inhibitor of a matrix metalloproteinase (MMP) such that the fatty acid inhibitor of the MMP causes the regression of a pre-existing aneurysm. Additionally, an implantable medical device is provided for implanting in a vessel wall of a patient comprising a structural support and a fatty acid inhibitor of an MMP.

### FIELD OF THE INVENTION

The present invention relates to the field of treatment of aneurysms. Specifically the present invention provides fatty acid compositions and medical devices for the inhibition of aneurysm growth. Additionally, methods are provided for the treatment of aneurysms using the fatty acid compositions and medical devices.

### BACKGROUND OF THE INVENTION

Aneurysms arise when a thinning, weakening section of an artery wall balloons out and are generally treated when the artery expands to more than 150% of its normal diameter. The most common and deadly of these occur in the aorta, the large blood vessel stretching from the heart to the lower abdomen. A normal aorta is between 1.6 to 2.8 centimeters wide; if an area reaches as wide as 5.5 centimeters, the risk of rupture increases such that surgery is recommended. Aneurysms are asymptomatic and they often burst before the patient reaches the hospital.

Aneurysms are estimated to cause approximately 32,000 deaths each year in the United States. Additionally, aneurysm deaths are suspected of being underreported because sudden unexplained deaths, about 450,000 in the United States alone, are often simply misdiagnosed as heart attacks or strokes while many of them may be due to aneurysms. Aneurysms most often occur in the aorta, the largest artery in the body. Most aortic aneurysms, approximately 15,000/year, involve the abdominal aorta while approximately 2,500 occur in the chest. Cerebral aneurysms occur in the brain and present a more complicated case because they are more difficult to detect and treat, causing approximately 14,000 U.S. deaths per year. Aortic aneurysms are detected by standard ultrasound, computerized tomography (CT) and magnetic resonance imaging (MRI) scans and the increased use of these scanning techniques for other diseases has produced an estimated 200% increase in the diagnosis of intact aortic aneurysms. Approximately 200,000 intact aortic aneurysms are diagnosed each year due to this increased screening alone.

U.S. surgeons treat approximately 50,000 abdominal aortic aneurysms (AAA) each year, typically replacing the abnormal section with a plastic or fabric graft in an open surgical procedure. A less-invasive procedure that has more recently been used is the stent graft which threads a compressed tubular device to the aneurysm and is expected to span the aneurysm to provide support without replacing a section of the aorta. A vascular graft containing a stent (stent graft) is placed within the artery at the site of the aneurysm and acts as a barrier between the blood and the weakened wall of the artery, thereby decreasing pressure on the artery. This less invasive approach of stent grafting aneurysms can decrease the morbidity seen with conventional aneurysm repair. Additionally, patients whose multiple medical comorbidities make them excessively high risk for conventional aneurysm repair are candidates for stent grafting. Stent grafts have also emerged as a new treatment for a related condition, acute blunt aortic injury, where trauma causes damage to the artery. The abdominal aorta between the renal artery and the iliac branch is the most susceptible arterial site to aneurysms. However, stent grafts do not treat the aneurysm directly.

Degradation of extracellular matrix proteins has been observed in aneurysms, especially AAA, including the destruction of the medial elastic lamellae, chronic inflammation within the outer aortic wall and medial neovascularization. Increased local production of connective tissue proteinases, including serine proteinases, plasminogen activators, and matrix metalloproteases (MMPs), has been documented in aneurysm tissue and may contribute to the degradation of structurally important extracellular matrix proteins in aneurysms. The MMPs are enzymes involved in physiological processes including embryogenesis, angiogenesis, reproductive function and bone resorption. They are also associated with accelerated breakdown of connective tissues found in arthritis, skin diseases, metastases of malignant tumors and aneurysms. The balance between the production and activation of MMPs and their inhibition by tissue inhibitors of metalloproteinases is an important aspect of disease progression. Therefore, inhibition of MMPs has been the subject of intense interest as a therapeutic target for a variety of diseases and disorders. Compounds have been identified which are effective on certain MMPs, such as gelatinases or collagenases, but they do not necessarily inhibit just one class of enzyme. For example, antibiotics such as tetracycline, gentamicin and cefalothin, inhibit the activity of collagenases originating from a variety of sources.

Fatty acids including, but not limited to, elaidic, oleic, eicosapentaenoic and dihydrolipoic acids, have been reported to inhibit MMPs. Oleic acid (cis-9-octadenanoic acid) reduces the activity of gelatinase A (MMP-2), which is secreted from cells as a zymogen and then activated to degrade extracellular matrix components. Fatty acids contribute to the regulation of extracellular matrix breakdown by inhibiting gelatinases A and B.

Inhibitors of MMPs have been proposed as treatments for aneurysms as disclosed in U.S. Pat. No. 5,834,449 to Thompson and Golub. This patent discloses the use of tetracycline and tetracycline derivatives for the treatment of aneurysms due to their ability to inhibit MMPs.

Therefore there is a need for methods and compositions which can treat the aneurysm using a pharmacologic approach targeted at inhibiting extracellular matrix degradation.

### SUMMARY OF THE INVENTION

The present invention provides compositions and related methods of treating aneurysms in vascular tissues, to prevent their inception and growth and to induce regression of established aneurysms by the administration of fatty acid inhibitors of matrix metalloproteinases directly to the aneurysm site.

In one embodiment according to the present invention, a method of treating an aneurysm in a patient in need thereof is provided comprising delivering to a treatment site an effective amount of a fatty acid inhibitor of a matrix metalloproteinase (MMP) such that the fatty acid inhibitor of a matrix metalloproteinase causes the regression of a pre-existing aneurysm. The treatment site is an aneurysm site such as an aneurysm sac or a peri-sac region.

In another embodiment, the fatty acid inhibitor of an MMP is elaidic acid or oleic acid.

In yet another embodiment, the fatty acid inhibitor of an MMP is associated with a carrier substrate. The carrier substrate is selected from the group consisting of biocompatible polymers, biocompatible biodegradable polymers, hydrogels and biological polymers. The carrier substrate and the fatty acid inhibitor of an MMP take the form selected from the group consisting of a pellet, a gel, a stent and a mesh. The pellet is selected from the group consisting of particles, microparticles and microbeads.

In one embodiment, the method further comprises administering a stent graft to the treatment site. In another embodiment, the fatty acid inhibitor of an MMP is delivered to the treatment site using an injection catheter.

The present invention also provides an implantable medical device for implantion in the vessel wall of a patient comprising a structural support and a fatty acid inhibitor of a matrix metalloproteinase. In one embodiment, the fatty acid inhibitor of an MMP is elaidic acid or oleic acid. In another embodiment, the structural support is selected from the group consisting of a stent, a stent graft and a mesh.

In another embodiment of the implantable medical device, the fatty acid inhibitor of an MMP is coated on the vessel wall-contacting surface of the medical device. In yet another embodiment, the fatty acid inhibitor of an MMP is coated on the vessel wall-contacting surface of the medical device in a biocompatible polymer coating.

### BRIEF DESCRIPTION OF THE FIGURES

FIG. 1 depicts a fully deployed stent graft with an exterior metal scaffolding as used in one embodiment according to the present invention.

FIGS. 2a-b depict a stent graft delivery catheter containing a multilumen injection catheter for coating the stent graft with at least one fatty acid inhibitor of an matrix metalloproteinase (MMP) immediately prior to deployment in accordance with the teachings according to the present invention.

FIGS. 3a-b depict a method of injection of a fatty acid inhibitor of an MMP directly into the aneurysm sac after deployment of a stent graft in accordance with the teachings according to the present invention.

FIGS. 4a-c depict an alternate method of injection of a fatty acid inhibitor of an MMP directly into the aneurysm sac after deployment of a stent graft in accordance with the teachings according to the present invention.

### DEFINITION OF TERMS

Prior to setting forth embodiments according to the present invention, it may be helpful to an understanding thereof to set forth definitions of certain terms that will be used hereinafter:

Aneurysm: As used herein “aneurysm” shall include a weak section of an artery wall in an animal and any abnormal dilatations of blood vessels.

Biocompatible: As used herein “biocompatible” shall include any material that does not cause injury or death to the animal or induce an adverse reaction in an animal when placed in intimate contact with the animal's tissues. Adverse reactions include inflammation, infection, fibrotic tissue formation, cell death, or thrombosis.

Biodegradable: As used herein, “biodegradable” shall include polymeric compositions that are biocompatible and subject to being broken down in vivo through the action of normal biochemical pathways. From time-to-time bioresorbable and biodegradable may be used interchangeably, however they are not coextensive. Biodegradable polymers may or may not be reabsorbed into surrounding tissues, however all bioresorbable polymers are considered biodegradable. The biodegradable polymers used in accordance with the present invention are capable of being cleaved into biocompatible byproducts through chemical- or enzyme-catalyzed hydrolysis.

Carrier substrate: As used herein, “carrier substrate” shall include materials which provide a vehicle for delivering bioactive materials to a treatment site or provide a solid substrate incorporating the bioactive materials. Non-limiting examples of carrier substrates are polymers including, but not limited to biodegradable polymers, biodegradable biocompatible polymers, hydrogels, biological polymers and in situ cross-linkable polymers.

Controlled release: As used herein, “controlled release” shall include the release of a bioactive material from a medical device surface at a predetermined rate. Controlled release implies that the bioactive material does not come off the medical device surface sporadically in an unpredictable fashion and does not “burst” off of the device upon contact with a biological environment (also referred to herein as first order kinetics) unless specifically intended to do so. However, the term “controlled release” as used herein does not preclude a “burst phenomenon” associated with deployment.

In some embodiments according to the present invention an initial burst of bioactive materials may be desirable followed by a more gradual release thereafter. The release rate may be steady state (commonly referred to as “timed release” or zero order kinetics), that is the bioactive material is released in even amounts over a predetermined time (with or without an initial burst phase) or may be a gradient release. A gradient release implies that the concentration of bioactive material released from the device surface changes over time.

Bioactive material(s): As used herein, “bioactive material(s)” shall include any bioactive compound, drug, therapeutic agent or composition having a biological effect in an animal. Exemplary, non limiting examples include small molecules, peptides, proteins, hormones, DNA or RNA fragments, genes, cells, genetically-modified cells, cell growth promoting compositions, inhibitors of matrix metalloproteinase, fatty acids and autologous platelet gel.

Stent graft: As used herein, “stent graft” shall include a tube comprising fabric, metal, composite, and/or derivations and combinations of these materials that reinforces a weakened portion of a vessel (in one instance, an aneurysm).

Treatment Site: As used herein, “treatment site” refers to an aneurysm site including the aneurysm sac and the peri-sac area.

### DETAILED DESCRIPTION

The present invention provides compositions and related methods of treating aneurysms in vascular tissues, to prevent their inception and growth and to induce regression of established aneurysms by the administration of fatty acid inhibitors of matrix metalloproteinases directly to the aneurysm site. The compositions and methods can also be applied for the prophylaxis, treatment and management of any abnormal dilatation of blood vessels, including aneurysms. Such dilatations are often considered to be predictive of aneurysm formation or diagnostic of incipient aneurysms and can be treatable or preventable using the compositions and methods according to the present invention.

Degradation of extracellular matrix proteins is an important histologic finding in aneurysms. The matrix metalloproteinases (MMPs) are enzymes involved in physiological processes including embryogenesis, angiogenesis, reproductive function and bone resorption. They are also associated with accelerated breakdown of connective tissues found in arthritis, skin diseases, metastases of malignant tumors and aneurysms. The balance between the production and activation of MMPs and their inhibition by tissue inhibitors of metalloproteinases is an important aspect of disease progression. Therefore, inhibition of MMPs has been the subject of intense interest as a therapeutic target for a variety of diseases and disorders. Compounds have been identified which are effective on certain MMPs, such as gelatinases or collagenases, but they do not necessarily inhibit just one class of enzyme. For example, antibiotics such as tetracycline, gentamicin and cefalothin, inhibit the activity of collagenases originating from a variety of sources.

Inhibitors of MMPs have been proposed as treatments for aneurysms as disclosed in U.S. Pat. No. 5,834,449 to Thompson and Golub. This patent discloses the use of tetracycline and tetracycline derivatives for the treatment of aneurysms due to their ability to inhibit MMPs.

Fatty acids including, but not limited to, elaidic, oleic, eicosapentaenoic and dihydrolipoic acids, have been reported to inhibit MMPs. Oleic acid (cis-9-octadenanoic acid) reduces the activity of gelatinase A (MMP-2), which is secreted from cells as a zymogen and then activated to degrade extracellular matrix components. Fatty acids contribute to the regulation of extracellular matrix breakdown by inhibiting gelatinases A and B.

As discussed briefly above, an aneurysm is swelling, or expansion of the blood vessel lumen at a defined point and is generally associated with a vessel wall defect. Aneurysms are often a multi-factorial asymptomatic vessel disease that if left unchecked may result in spontaneous rupture, often with fatal consequences. Previous methods to treat aneurysms involved highly invasive surgical procedures where the affected vessel region was removed and replaced with a synthetic graft that was sutured in place. However, this procedure is extremely risky and generally only employed in otherwise healthy vigorous patients who are expected to survive the associated surgical trauma. Elderly and more feeble patients were not candidates for many aneurysmal surgeries and remained untreated and thus at continued risk for sudden death.

In order to overcome the risks associated with invasive aneurysmal surgeries, stent grafts that can be deployed with a cut down procedure or percutaneously using minimally invasive procedures were developed. Essentially, a catheter having a stent graft compressed and fitted into the catheter's distal tip is advanced through an artery to the aneurysmal site. The stent graft is then deployed within the vessel lumen juxtaposed to the weakened vessel wall forming an inner liner that insulates the aneurysm from the body's hemodynamic forces thereby reducing, or eliminating the possibility of rupture. The size and shape of the stent graft is matched to the treatment site's lumen diameter and aneurysm length. Moreover, branched grafts are commonly used to treat abdominal aortic aneurysms that are located near the iliac branch.

While the implantation of a stent graft at an aneurysm site provides a physical support for the damaged section of vessel wall, it does not treat the aneurysmal tissue. Additionally, stent grafts often fail due to flow of blood between the stent graft and the vessel wall (endoleak), rather than through the lumen of the stent graft, causing migration and failure of the graft. Implantation of a stent graft and a bioactive material directly at the aneurysm site provides both structural support for the weakened vessel and treatment and management of the aneurysm through pharmacologic intervention.

Therefore, the present invention discloses compositions, medical devices and methods comprising delivery of fatty acid inhibitors of MMPs to treatment sites for the treatment of aneurysms. One embodiment according to the present invention provides a therapeutic composition comprising at least one fatty acid inhibitor of an MMP including, but not limited to, elaidic and oleic acids, associated with a medical device and/or a carrier substrate for the treatment of aneurysms.

The fatty acid inhibitors of an MMP according to the present invention are delivered to an aneurysm site along with a suitable medical device and/or carrier substrate such that the compositions are localized at the treatment site for a period of time. Delivery of solubilized bioactive materials to sites within the vasculature has been attempted using weeping balloon and injection catheters without success. These approaches have not been successful because blood flow quickly flushes the bioactive material downstream and away from the treatment site. For this reason, methods of delivery of bioactive materials coated on stent grafts or administered in a gel were developed. However, for delivery of bioactive materials to aneurysm sites a third treatment regimen is also possible. Once a stent graft has been deployed at an aneurysm site, bioactive materials, either soluble or immobilized within a carrier substrate, can be delivered to the aneurysm sac between the implanted stent graft and the aneurysm wall.

In one embodiment according to the present invention, the fatty acid inhibitors of an MMP are administered to the aneurysm site on a stent graft. Bioactive material-coated stent grafts are manufactured by spraying, rolling or dipping the stent graft in a solution containing the bioactive material or by coating the stent with a bioactive material-containing material. Alternatively, depending on the characteristics of the bioactive material, it may be desirable to coat the stent graft with the composition dispersed in a suitable polymer coating. Biocompatible and hemocompatible polymers, including, but not limited to, poly(ethylene glycol) (PEG)-containing polymers, PCL (Polycaprolactone), PGA (Polyglycolide), PLA (Poly-L-lactide) Other bio/environmentally degradable polymers include poly(hydroxyalkanoate)s of the PHB-PHV class, additional poly(ester)s, hyaluronic acid (HA) and other natural polymers, particularly, modified poly(saccharide)s, e.g., starch, cellulose, and chitosan are known to those of skill in the polymer coating art.

Stent grafts generally comprise a metal scaffolding having a biocompatible covering such as Dacron® (E.I. du Pont de Nemours & Company, Wilmington, Del.) or a fabric-like material woven from a variety of biocompatible polymer fibers. Other embodiments include extruded sheaths and coverings. The scaffolding is generally on the luminal wall-contacting surface of the stent graft and directly contacts the vessel lumen. The sheath material is stitched, glued or molded onto the scaffold. In other embodiments, the scaffolding may be on the stent graft's blood flow contacting surface or interior. When a self-expanding stent graft is deployed from the delivery catheter, the scaffolding expands to fill the lumen and exerts circumferential force against the lumen wall. This circumferential force is generally sufficient to keep the stent-graft from migrating, thus preventing endoleak. However, stent migration and endoleak may occur in vessels that have irregular shapes or are shaped such that they exacerbate hemodynamic forces within the lumen. Stent graft migration refers to a stent graft moving from the original deployment site, usually in the direction of the blood flow. Stent graft migration may result in endoleak, the aneurysmal sac being exposed to blood pressure again and an increasing risk of rupture.

In one embodiment according to the present invention, the stent graft is pre-coated with at least one a fatty acid inhibitor of an MMP prior to loading into a delivery catheter and deploying to a treatment site. In another embodiment the fatty acid inhibitor of an MMP is coated on the stent graft in a polymer coating, wherein the polymer coating is selected from the group consisting of biocompatible polymers, biocompatible biodegradable polymers, hydrogels and biological polymers.

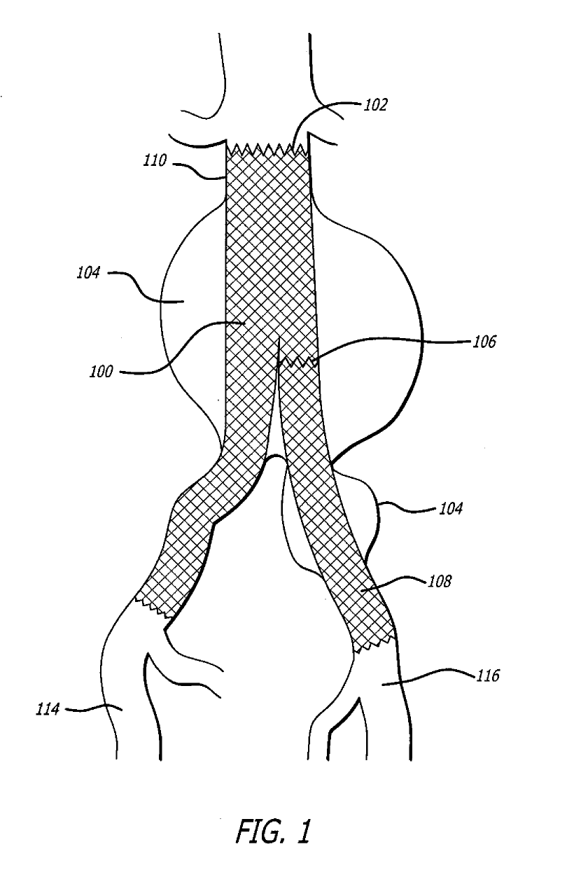

In an exemplary stent deployment protocol wherein the aneurysm site is the site of an abdominal aortic aneurysm, a vascular stent graft 100 is fully deployed through the left iliac artery 114 to an aneurysm site 104 (FIG. 1). Stent graft 100 has a distal end 102 and an iliac leg 108 to anchor the stent graft in the iliac artery 116. Stent graft 100 is deployed first in a first delivery catheter and the iliac leg 108 is deployed in a second delivery catheter. The stent graft 100 and iliac leg 108 are joined with an approximate 2 cm overlap of the two segments 106.

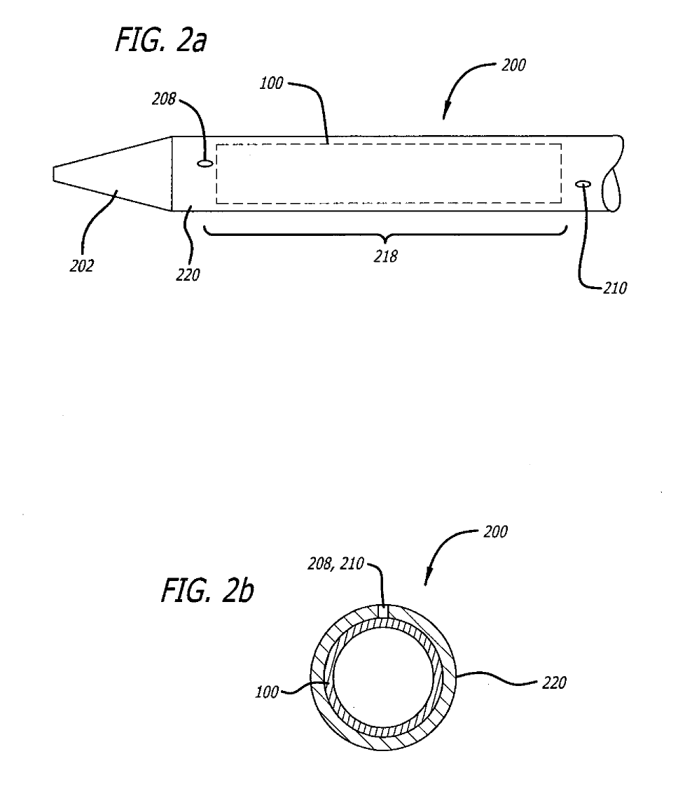

In one embodiment, at least one fatty acid inhibitor of an MMP is applied to a stent graft while the stent graft is disposed within the delivery device, prior to the deployment of the stent graft. An exemplary, non-limiting delivery device suitable for coating a stent graft is depicted in FIG. 2a. The stent graft is provided “pre-loaded” into a delivery catheter and the fatty acid composition is applied to the stent graft while the stent graft is disposed within the delivery catheter. In FIG. 2a, stent graft 100 is radially compressed to fill the stent graft chamber 218 in the distal end 202 of delivery catheter 200. The stent graft 100 is covered with a retractable sheath 220. Catheter 200 has two injection ports 208 and 210 for delivering the fatty acid composition to the compressed stent graft. In this embodiment, the fatty acid composition is injected through either or both of injection ports 208 and 210 to wet stent graft 100. Stent graft 100 is then deployed to the treatment site as depicted in FIG. 1. FIG. 2b depicts a cross-sectional view of stent graft 100 loaded into the delivery catheter 200 and the delivery catheter's retractable sheath 220 illustrating an injection port 208 for delivering the fatty acid composition to stent graft 100.

In yet another embodiment, a composition comprising a fatty acid inhibitor of an MMP and a carrier substrate is provided, wherein in the carrier substrate immobilizes the composition at the aneurysm site and prevents the vascular flow from washing the fatty acid away.

Carrier substrates provide a vehicle or structural support for delivery of bioactive materials and provide for controlled-release of the bioactive materials at the treatment site. Carrier substrates suitable for the delivery of fatty acid inhibitors of an MMP to an aneurysm site include, but are not limited to, biocompatible polymers, hydrogels, collagen, Alginate, hyaluronic acid (HA) and chitosan.

Suitable biocompatible polymers can be biodegradable, bioerodable, water soluble, cross-linkable or other polymer types. Additionally, the polymers can take the form of pellets such as particles, microparticles or microbeads, or gels, biodegradable or bioerodable stents or meshes.

In one embodiment, the carrier substrate is a biocompatible polymer. The biocompatible polymer and at least one fatty acid inhibitor of an MMP are formed into pellets, the pellets are implanted at the treatment site and the fatty acid inhibitor of an MMP released from the pellets over a period of time. The biocompatible polymers chosen for this embodiment are selected to provide a predictable release rate of fatty acids to the treatment site. Such release rate can be, without limitation, rapid, as in a “burst” of fatty acid release from the polymer, or slow wherein the fatty acids are released over a period of days, weeks or months after administration. Non-limiting examples of biocompatible polymers suitable for releasing the fatty acid inhibitors of an MMP according to the present invention include, biocompatible polymers, hydrogels, collagen, Alginate, hyaluronic acid (HA) and chitosan. The biocompatible polymer can either be impregnated with the fatty acids or the fatty acids can be grafted. A graft copolymer has polymer chains of one kind growing out of the sides of polymer chains with a different chemical composition on the biocompatible polymer. Cross-links are covalent bonds linking one polymer chain to another.

In another embodiment, the carrier substrate is a biocompatible, biodegradable polymer. The biocompatible, biodegradable polymer and at least one fatty acid inhibitor of an MMP are formed into pellets, the pellets are implanted at the treatment site and the fatty acid released from the pellets over a period of time. The biodegradable polymer chosen for this embodiment is selected to provide a predictable degradation profile such that the fatty acids are released from the polymer as the polymer degrades. The degradation profile of the biodegradable polymer is chosen to provide the release rate desired for a specific use. Non-limiting examples of biocompatible polymers suitable for releasing the fatty acid inhibitors of an MMP according to the present invention include, biocompatible polymers, hydrogels, collagen, Alginate, hyaluronic acid (HA) and chitosan. Exemplary, non-limiting polymers are disclosed in co-owned and co-pending U.S. patent application Ser. No. 10/970,171 and U.S. Provisional Patent Application No. 60/711,895, which are incorporated herein by reference for all they contain regarding biodegradable and biocompatible polymers. The biocompatible, biodegradable polymers can either be impregnated with the fatty acids or the fatty acids can be grafted on the biocompatible, biodegradable polymer.

Biodegradable polymers, either synthetic or natural, are capable of being cleaved into biocompatible byproducts through chemical- or enzyme-catalyzed hydrolysis. This biodegradable property makes it possible to implant them into the body without the need of subsequent surgical removal. Moreover, bioactive materials formulated with these polymers can be released in a controlled manner, by which the bioactive material concentration in the target site is maintained within the therapeutic window. The release rates of the bioactive materials from the biodegradable polymer can be controlled by a number of factors, such as biodegradation rate, physiochemical properties of the polymers and bioactive materials, thermodynamic compatibility between the polymer and bioactive material and the shape of the resulting polymer-containing composition.

In yet another embodiment, the carrier substrate is a hydrogel. Hydrogels are three dimensional networks of hydrophilic polymer chains that are cross-linked through either chemical or physical bonding. Because of the hydrophilic nature of the polymer chains, hydrogels absorb water to swell in the presence of abundant water. The release of bioactive materials from hydrogels can be controlled by the cross-linking density, polymer molecular weight, the nature of the degradable linkages and where the bioactive material is covalently incorporated or physically trapped within the polymer matrix. Hydrogels can be formulated so that they are liquids at room temperature and gels at physiologic temperature thereby making them particularly suited to delivery to vascular treatment sites using injection catheters. Non-limiting examples of hydrogels suitable for releasing the a fatty acid inhibitors of an MMP include, without limitation, PEG-based hydrogels, and PEO, PVP and HA based hydrogels. In this embodiment, the at least one fatty acid inhibitor of an MMP is impregnated in or grafted onto the hydrogel, the fatty acid/hydrogel compositions are delivered to the treatment site and the fatty acid released from the hydrogel over a period of time.

In still another embodiment, the carrier substrate is a biological polymer including, but not limited to, collagen, aginate and hyaluronic acid. The biological polymers can either be impregnated within the fatty acid inhibitors of an MMP according to the present invention or the fatty acids can be grafted on the biological polymers. The fatty acid/biological polymer compositions are delivered to the treatment site and the fatty acid released from the biological polymer over a period of time.

In another embodiment, the carrier substrate is an in situ cross-linkable polymer. An in situ cross-linkable polymer is delivered to a treatment site in a non-cross-linked fluid state and undergoes a physical or chemical reconfiguration at the treatment site such that the polymer material assumes the desired shape or configuration. The physical or chemical reconfiguration can be induced by application of a cross-linking agent in situ or by application of a physical stimulus such as, but not limited to, light. In this embodiment, the fatty acid inhibitors of an MMP according to the present invention are delivered to the treatment site associated with the non-cross-linked polymeric material, allowed to coat the aneurysm sac and then cross-linked to form a solid substrate within the aneurysm sac. As with the other polymeric materials disclosed above, the fatty acids are released from the polymer at rates governed by the composition and configuration of the polymer.

The fatty acid inhibitors of an MMP/carrier substrate composition are delivered to the aneurysm site using delivery devices known to persons skilled in the art including, but not limited to, injection catheters. Alternatively, the fatty acid compositions can be delivered to the treatment site using injection catheters specifically designed to administer bioactive materials within a vessel while maintaining nearly-constant pressure in the treatment site. These injection catheters maintain nearly-constant pressure by having with at least one injection lumen and at least one exit lumen. As a bioactive material and/or a carrier substrate is administered within a vessel through the one or more injection lumens, displaced blood or other fluids in the area that would normally contribute to an increase in internal pressure at the administration site, instead leave the site through an exit port and lumen. Thus, nearly-constant pressure at the administration site can be maintained despite the addition of bioactive materials within the confined space. This maintenance of nearly-constant pressure decreases the risk that the introduction of bioactive materials at a treatment site would lead to vessel rupture. An injection catheter useful for maintaining nearly-constant pressure at a treatment site is disclosed in co-owned and co-pending U.S. patent application Ser. No. 11/276,517, the disclosure of which is incorporated herein by reference for all it contains regarding injection catheters.

In yet another embodiment, the carrier substrate, associated with the fatty acid inhibitors of an MMP according to the present invention, are formed into an implantable vascular device that is delivered to a treatment site and expanded to fill the treatment site. Non-limiting examples of implantable medical devices which can be formed from the carrier substrate/fatty acid compositions are stents, stent grafts and meshes.

In yet another embodiment, the fatty acid inhibitors of an MMP/carrier substrate compositions are administered to a treatment site with a stent graft such that the fatty acid/carrier substrate composition is confined between the outer wall of the stent graft and the inner wall of the vessel.

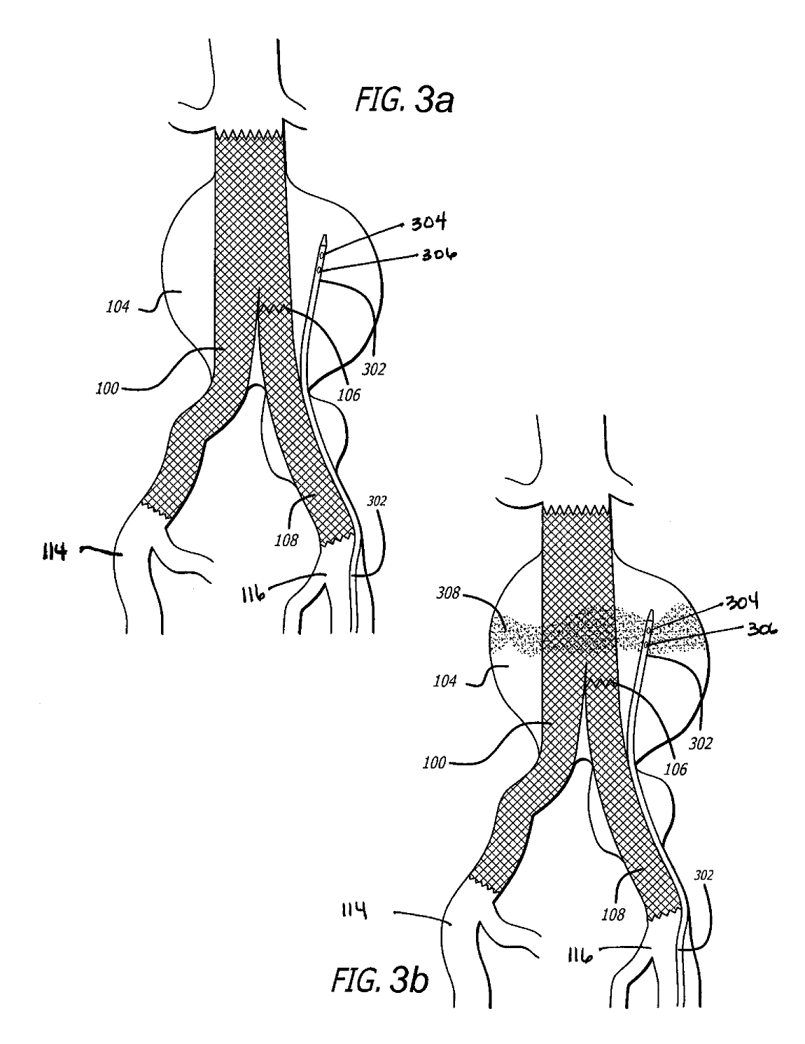

In this embodiment stent graft 100 is deployed to the treatment site via the left iliac artery 114. An injection catheter 302 is also deployed to the aneurysm sac through the right iliac artery 116 (FIG. 3a). Injection catheter 302 has injection ports 304 and 306 through which the fatty acid composition may be deposited. The fatty acid composition is injected between the vessel lumen wall and the stent graft within the aneurysm sac 308 (FIG. 3b). The injection catheter 302 is then retrieved.

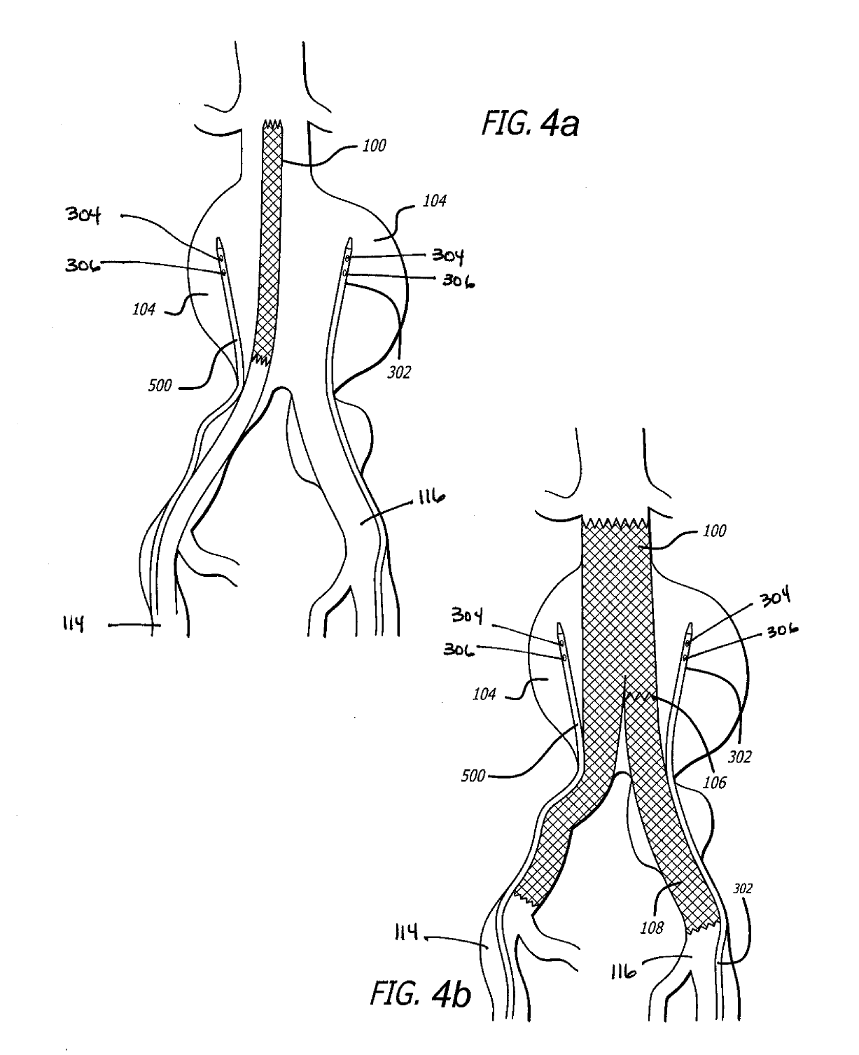

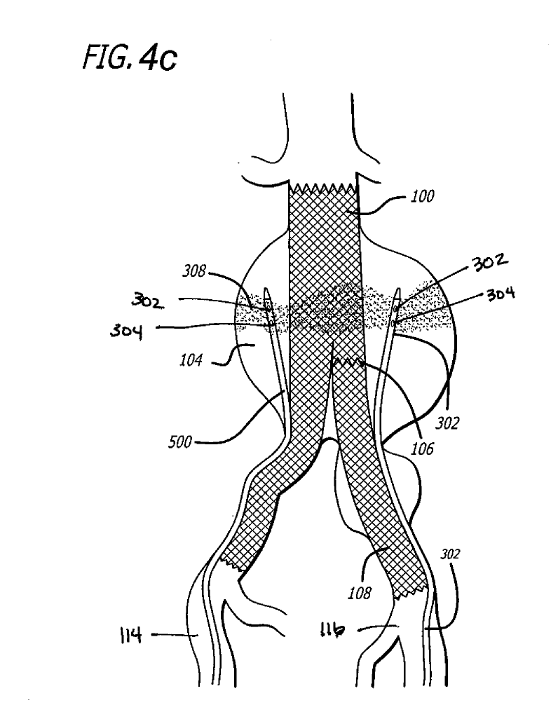

In an alternative embodiment, more than one injection catheter can be used to deliver the fatty acid composition to the aneurysm sac 104 (FIG. 4a, b & c). As previously described, stent graft 100 is radially compressed to fill a stent graft chamber of a stent delivery catheter which is then deployed to the treatment site via the left iliac artery 114 (FIG. 4a). Multiple single lumen or multilumen injection catheters 302 and 500 may also be deployed to the aneurysm sac 104 through the right iliac artery 116 and left iliac artery 114. Injection catheters 302 and 500 have injection ports through which the fatty acid composition may be deposited. The delivery catheter is removed and the iliac limb 108 is deployed as in FIG. 1 while injection catheters 302 and 500 remain in place (FIG. 4b) with their injection ports 304 and 306 in the aneurysm sac 104. The iliac limb segment of the stent graft seals the aneurysm sac at the proximal end. The fatty acid composition 308 is injected between the vessel lumen wall and the stent graft within the aneurysm sac (FIG. 4c). The injection catheters 302 and 500 are then retrieved.

The field of medical device coatings is well established and methods for coating stent grafts with bioactive materials, with or without added polymers, are well known to those of skill in the art. Non-limiting examples of coating procedures include spraying, dipping, waterfall application, heat annealing, etc. The amount of coating applied to a stent graft can vary depending upon the desired effect of the compositions contained within the coating. The coating may be applied to the entire stent graft or to a portion of the stent graft. Thus, various bioactive material coatings applied to stent grafts are within the scope of embodiments according to the present invention.

The appropriate anti-aneurysmal dosage of fatty acid inhibitor of MMPs is selected by the practitioner such as a physician, who is guided by skill and knowledge in the field and comprises an amount of the fatty acid inhibitor of MMPs which induces an anti-aneurysmal effect at the aneurysm site. An anti-aneurysmal effect is prevention of the formation of an aneurysm in a blood vessel, inhibition of the progression or induction of the regression of an established (pre-existing) aneurysm. The compositions, devices and methods according to the present invention are effective in resisting the formation of such aneurysms, and are helpful in causing the regression of pre-existing aneurysms to cause the involved blood vessel (aneurysmal tissue) to return to a safer, normal or near normal state.

Unless otherwise indicated, all numbers expressing quantities of ingredients, properties such as molecular weight, reaction conditions, and so forth used in the specification are to be understood as being modified in all instances by the term “about.” Accordingly, unless indicated to the contrary, the numerical parameters set forth in the following specification are approximations that may vary depending upon the desired properties sought to be obtained. Notwithstanding that the numerical ranges and parameters are approximations, the numerical values set forth in the specific examples are reported as precisely as possible. Any numerical value, however, inherently contains certain errors necessarily resulting from the standard deviation found in their respective testing measurements.

The terms “a” and “an” and “the” and similar referents used are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided herein is intended to better illuminate embodiments according to the invention.

Groupings of alternative elements or embodiments according to the invention disclosed herein are not to be construed as limitations. Each group member may be referred to individually or in any combination with other members of the group or other elements found herein. It is anticipated that one or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

Embodiments of this invention are described herein. Of course, variations on those embodiments will become apparent to those of ordinary skill in the art upon reading the foregoing description unless otherwise indicated herein or otherwise clearly contradicted by context.

Furthermore, numerous references have been made to patents and printed publications throughout this specification. Each of the above cited references and printed publications are herein individually incorporated by reference in their entirety.

What is claimed is:

1. A method of treating an aneurysm in a patient in need thereof comprising:
delivering to a treatment site an effective amount of a fatty acid inhibitor of a matrix metalloproteinase (MMP) such that said fatty acid inhibitor of said matrix metalloproteinase causes the regression of a pre-existing aneurysm.

2. The method according to claim 1 wherein said treatment site is an aneurysm site such as an aneurysm sac or a peri-sac region.

3. The method according to claim 1 wherein said fatty acid inhibitor of said MMP is elaidic acid or oleic acid.

4. The method according to claim 1 wherein said fatty acid inhibitor of said MMP is associated with a carrier substrate.

5. The method according to claim 4 wherein said carrier substrate is selected from the group consisting of biocompatible polymers, biocompatible biodegradable polymers, hydrogels and biological polymers.

6. The method according to claim 4 wherein said carrier substrate and said fatty acid inhibitor of said MMP take a form selected from the group consisting of a pellet, a gel, a stent and a mesh.

7. The method according to claim 6 wherein said pellet takes a form selected from the group consisting of particles, microparticles and microbeads.

8. The method according to claim 1 further comprising administering a stent graft to said treatment site.

9. The method according to claim 1 or 4 wherein said fatty acid inhibitor is delivered to said treatment site using an injection catheter.

10. An implantable medical device for implanting in a vessel wall of a patient comprising:
a structural support and a fatty acid inhibitor of a matrix metalloproteinase (MM P).

11. The implantable medical device according to claim 10 wherein said fatty acid inhibitor of said MMP is elaidic acid or oleic acid.

12. The implantable medical device according to claim 10 wherein said structural support is selected from the group consisting of a stent, a stent graft and a mesh.

13. The implantable medical device according to claim 10 wherein said fatty acid inhibitor of said MMP is coated on the vessel wall-contacting surface of said medical device.

14. The implantable medical device according to claim 13 wherein said fatty acid inhibitor of said MMP is coated on the vessel wall-contacting surface of said medical device in a biocompatible polymer coating
